SEQUENCE LISTING (5 sequences)

SEQ ID NO: 1 - DNA, 523 bp, Homo sapiens
  CDS (8)...(517)
cctcgagatgaagaccctgttcctgggtgtcacgctcggcctggccgctgccctgtccttcaccctggaggaggaggatatcacagggacctggtacgtgaaggccatggtggtcgataaggactttccggaggacaggaggcccaggaaggtgtccccagtgaaggtgacagccctgggcggtgggaagttggaagccacgttcaccttcatgagggaggatcggtgcatccagaagaaaatcctgatgcggaagacggaggagcctggcaaatacagcgcctatgggggcaggaagctcatgtacctgcaggagctgcccaggagggaccactacatcttttactgcaaagaccagcaccatgggggcctgctccacatgggaaagcttgtgggtaggaattctgataccaaccgggaggccctggaagaatttaagaaattggtgcagcgcaagggactctcggaggaggacattttcacgcccctgcagacgggaagctgcgttcccgaacacggatcc

SEQ ID NO: 2 - protein, 170 aa, Homo sapiens
MKTLFLGVTLGLAAALSFTLEEEDITGTWYVKAMVVDKDFPEDRRPRKVSPVKVTALGGGKLEATFTFMREDRCIQKKILMRKTEEPGKYSAYGGRKLMYLQELPRRDHYIFYCKDQHHGGLLHMGKLVGRNSDTNREALEEFKKLVQRKGLSEEDIFTPLQTGSCVPEH

SEQ ID NO: 3 - DNA, 811 bp, Homo sapiens
  CDS (99)...(584)
catccctctggctccagagctcagagccacccacagccgcagccatgctgtgcctcctgctcaccctgggcgtggccctggtctgtggtgtcccggccatggacatcccccagaccaagcaggacctggagctcccaaagttggcagggacctggcactccatggccatggcgaccaacaacatctccctcatggcgacactgaaggcccctctgagggtccacatcacctcactgttgcccacccccgaggacaacctggagatcgttctgcacagatgggagaacaacagctgtgttgagaagaaggtccttggagagaagactgggaatccaaagaagttcaagatcaactatacggtggcgaacgaggccacgctgctcgatactgactacgacaatttcctgtttctctgcctacaggacaccaccacccccatccagagcatgatgtgccagtacctggccagagtcctggtggaggacgatgagatcatgcagggattcatcagggctttcaggcccctgcccaggcacctatggtacttgctggacttgaaacagatggaagagccgtgccgtttctagctcacctccgcctccaggaagaccagactcccacccttccacacctccagagcagtgggacttcctcctgccctttcaaagaataaccacagctcagaagacgatgacgtggtcatctgtgtcgccatccccttcctgctgcacacctgcaccattgccatggggaggctgctccctgggggcagagtctctggcagaggttattaataaacccttggagcatg

SEQ ID NO: 4 - protein, 162 aa, Homo sapiens
MDIPQTKQDLELPKLAGTWHSMAMATNNISLMATLKAPLRVHITSLLPTPEDNLEIVLHRWENNSCVEKKVLGEKTGNPKKFKINYTVANEATLLDTDYDNFLFLCLQDTTTPIQSMMCQYLARVLVEDDEIMQGFIRAFRPLPRHLWYLLDLKQMEEPCRF

SEQ ID NO: 5 - protein, 16 aa, Artificial Sequence
  Peptide linker.
GGSGGSGGGGSGGGGS

(19) United States
(12) Patent Application Publication
(10) Pub. No.: US 2002/0098497 A1
(43) Pub. Date: Jul. 25, 2002

(54) Use of human phermone polypeptides

(76) Inventors: Si Lok, Seattle, WA (US); Donald C. Foster, Lake Forest Park, WA (US); James L. Holloway, Seattle, WA (US)

Correspondence Address: ZymoGenetics, Inc., 1201 Eastlake Avenue East, Seattle, WA, 98102, US

(21) Appl. No.: 09/951,845

(22) Filed: Sep. 12, 2001

Related U.S. Application Data
(60) Provisional application No. 60/232,218, filed on Sep. 13, 2000.

(51) Int. Cl. C12Q 1/68; G01N 33/53

(52) U.S. Cl. 435/6; 435/7.1

(57) ABSTRACT

Human phermones may be used to alleviate anxiety, promote beneficial moods, and to alter hypothalamic functions, such as satiety, energy balance, and reproductive biology. The present invention provides methods for using Zlipo1 and glycodelin as pheromone polypeptides.

### CROSS-REFERENCE TO RELATED APPLICATIONS

This application claims the benefit of U.S. Provisional application No. 60/232,218 (filed Sep. 13, 2000), the contents of which are incorporated by reference.

### TECHNICAL FIELD

The present invention relates generally to new methods of using two human lipocalin proteins. In particular, the present invention relates to methods of using Zlipo1 and glycodelin as phermone polypeptides.

### BACKGROUND OF THE INVENTION

Olfaction is an ancient sense, rudiments of which can be found in the most primitive single-celled organisms (see generally, Tirindelli et al., TINS 11:482 (1998); Keverne, Science 286:716 (1999); Liman, Current Opinion in Neurobiology 6:487 (1996); Buck, Cell 65:175 (2000)). The importance of this sense is exemplified by the fact that humans are capable of perceiving thousands of discrete odors, and that more than 1% of the genes in the human genome are devoted to olfaction. Olfaction has an aesthetic component that is capable of invoking emotion and memory leading to measured thoughts and response to the everyday environment. However, in some species, a diverse class of molecules, generally referred to as pheromones, can elicit innate and stereotyped behaviors that are likely to result from non-conscious perception.

At present, the majority of the identified pheromones are from insects. Many insect species produce potent volatile chemical compounds, which attract potential mates over long distances (Kaissling, Ann. Rev. Neurosci. 9:121 (1986); Masson and Mustaparta, Physiol. Rev. 70:199 (1990)). Synthetic versions of certain pheromones are used as chemo-attractants to control insect pests.

Members of the animal kingdom are also known to produce pheromones for intra-species communication. F-prostaglandins and steroids, for example, have been shown to induce sperm production and mating in fish (Stacey and Sorensen, Can. J. Zool. 64:2412 (1986); Sorensen et al., Biol. Reprod. 39:1039 (1988)). A family of pheromones comprised of dianeackerone-relalated steroidal esters has been found to play a role in nesting and mating in crocodiles (Whyte et al., Proc. Nat'l Acad. Sci. (USA) 96:12246 (1999); Yang et al., Proc. Nat'l Acad. Sci. (USA) 96:12251 (1999)). A series of nonvolatile saturated and monosaturated long-chain methyl ketones, and compounds containing squalene were shown to induce courtship behavior in garter snakes (Mason et al., Science 293:290 (1989)). Recently, a proteinaceous pheromone affecting female receptivity was isolated from a terrestrial salamander, and a peptide with female-attracting activity was identified in newts (Kikuyama et al., Science 267:1643 (1995); Rollmann et al., Science 285:1907 (1999)).

Mammalian pheromones have also been described. In mammals, the two pathways of olfactory perception are mediated by anatomically distinct sensory organs. The main olfactory epithelium recognizes everyday ordorants and certain phermones, whereas the vomeronasal organ specializes in the perception of pheromones (see, for example, Buck, Cell 65:175 (2000); Liman, Cur. Opin. Neurobio. 6:487 (1996); Tirindelli et al., Trends Neurosci. 11:482 (1998); Keverne, Science 286:716 (1999)). The main olfactory epithelium and the neuroepithelium of the vomeronasal organ contain sensory neurons that project axons to the brain (Belluscio et al., Cell 97:209 (1999); Rodriguez et al., Cell 97:199 (1999)). Sensory inputs from the main olfactory epithelium ultimately reach multiple regions of the brain, including the frontal cortex, which process the conscious perception of odors. In contrast, pheromone derived signals from the vomeronasal organ can bypass higher cognitive centers and are processed directly in regions of the amygdala and hypothalamus that have been implicated in the regulation of innate behavior, reproductive physiology, energy balance and other neuroendocrine responses.

Rodents provide useful experimental animals for studying pheromone action. One vehicle of olfactory chemo-signals in the mouse is urine, which mediates a variety of behavioral and physiological responses. The role of saliva in sexual communication has also been demonstrated in mice (Marchlewska et al., J. Chem. Ecol. 16:2817 (1990)). The endocrine effects primed by male mouse urine include: acceleration of female puberty onset, pregnancy block, attraction to females, aggression, estrus acceleration, and estrus synchronization. Pheromone signaling in mice is characterized by at least three components: (1) chemosensory receptors present in the vomeronasal organ, and for some classes of pheromones, by receptors within the main olfactory epithelium; (2) volatile pheromone ordorants; and (3) a high concentration of pheromone binding proteins in the male mouse urine. Volatile pheromone molecules in urine are bound to a group of carrier proteins known as the major urinary proteins (MUP). These proteins are thought to promote stability of the bound pheromone and to effect their sustained release from urine (Hurst et al., Anim. Behav. 55:1289 (1988)).

A number of rodent volatile pheromones in mouse urine that bind with MUPs were recently characterized. Two major volatile constituents of the male rodent preputial gland, E,E-alpha-farnesene and E-beta-farnesene, attract females and induce estrus (Jemiolo et al., Physiology & Behavior 50:1119 (1991); Ma et al., Chem. Senses 24:289 (1999)). Another urine phermone, 6-hydroxy-6-methyl-3-heptanone, accelerates puberty in female mice (Novotny et al., Chemistry & Biology 6:377 (1999)). Other volatile phermones found in rodent urine, thiazole (2-sec-butyl-4,5-dihydrothiazole) and brevicomin (2,3-dehydro-exo-brevicomin), function as attractants for females, inducers of estrous, and instigators of inter-male aggression (Jemiolo et al., Anim. Behav. 33:1114 (1985); Novotny et al., Proc. Nat'l Acad. Sci. (USA) 82:2059 (1985); Jemiolo et al., Proc. Nat'l Acad. Sci. (USA) 83:4576 (1986); Hurst et al., Anim. Behav. 55:1289 (1988); Novotny et al., Proc. R. Soc. Lond. B. Biol. Sci. 266:2017 (2000)).

The rodent pheromone carrier proteins are members of the lipocalin family of extracellular proteins (see, for example, Flower, FEBS Lett. 354:7 (1994); Flower, Biochem. J. 318:1 (1996)). Lipocalins are characterized by a single eight-stranded hydrogen-bonded anti-parallel β-barrel, which in some members encloses an internal ligand-binding-site (Lucke et al., Eur. J. Biochem. 266:1210 (1999)). One important function of the lipocalins is to control and modulate the transport of small hydrophobic regulatory molecules between cells (Flower, FEBS Lett. 354:7 (1994)). Other portions of the protein are known to interact with cell-surface receptors or soluble macromolecules, which further add to the complex biological functions of these proteins.

An important recent finding is that, in addition to being proteinaceous carriers of small volatile pheromones, certain lipocalins, without bound pheromone ligands, appear to have pheromone activity (Mucignat-Caretta et al., J. Physiol. 486:517 (1995)). Furthermore, a hexapeptide derived from the amino-terminus of murine major urinary proteins (MUP) is active in the assay (Clark et al., EMBO J. 4:3159 (1985); Mucignat-Caretta et al., J. Physiol. 486:517 (1995)). Recombinant aphrodisin, a lipocalin family member found in vaginal discharge, can induce investigatory and copulatory responses in male hamsters in the apparent absence of a ligand (Macrides, et al., Phyiol. Behav. 33:633 (1984); Singer et al., J. Biol. Chem. 261:13312 (1986); Henzel et al., J. Biol. Chem. 263:16682 (1988); Singer and Macrides, Chem. Senses 15:199 (1990)). Pheromone activity of recombinant aphrodisin, however, is enhanced in the presence of organic extracts of hamster vaginal discharge suggesting that an as yet unidentified lipophilic ligand, working in conjugation with the aphrodisin protein, is required for the full pheromone response (Singer and Macrides, Chem. Senses 15:199 (1990)). Polypeptides with pheromone activity are not without precedence. There are several reports of proteinaceous pheromones in amphibian species (Kikuyama et al., Science 267:1643 (1995); Lebioda et al., Nature 401:444 (1999); Rollmann et al., Science 285:1907 (1999)).

It appears the pheromone system has, in some cases, evolved to recognize and to respond to both the pheromone ligand and its lipocalin carrier protein. Consistent with this hypothesis is that many lipocalins have regions on their surface, which are believed to interact with cell surface receptors and other regulatory molecules (Bocskei et al., Nature 360:186 (1992)). Results from signal transduction experiments support the hypothesis that MUPs have an independent role in pheromone recognition. MUP ligands, brevicomin or dihydrothiazole, appear to activate only a small subset of neurons of the accessory olfactory bulb when compared with the ligand together with its MUP binding protein (Brennan et al., Neuro-Science 90:1463 (1999)). Other evidence comes from rat α-2-glubulin, an orthologous protein to murine MUP. Recombinant α-2-glubulin was found to activate G-protein subtype Go, whereas stimulation with the α-2-glubulin ligand alone resulted in the activation of G-protein, Gi, in vomeronasal organ membrane preparations (Krieger et al., J. Biol. Chem. 274:4655 (1999)). Together, these results not only show that the MUPs and their ligands have independent pheromone activity, but that they can also activate distinct signaling pathways within the vomeronasal organ.

Most, but not all, mammalian pheromone recognition is mediated by the vomeronasal organ, which resides within a blind pouch in the septum of the nose (see, for example, Stensaas et al., J. Steroid. Biochem. Mol. Biol. 39:553 (1991); Monti-Bloch et al., Annals New York Academy of Sciences 30:373 (1998); Trindelli et al., Trend Neurosci. 21:482 (1998); Keverne, Science 286:716 (1999)). Two distinct families of pheromone receptor genes, V1 and V2, are expressed in rodent vomeronasal neurons (Dulac and Axel, Cell 83:195 (1995); Herrada and Dulac, Cell 90:763 (1997); Matsunami and Buck, Cell 90:775 (1997); Ryba and Trindelli, Neuron 19:371 (1997); Dulac and Axel, Chem. Senses 23:467 (1998)). The V1 and V2 receptor genes comprised two novel families of seven-transmembrane domain G-protein coupled receptor proteins that are distinct from the odorant receptors expressed in the main olfactory epithelium or to other families of seven-transmembrane domain receptors (Buck and Axel, Cell 65:175 (1991)).

The V2 receptors are related to the metabotropic glutamate receptors, and have a large N-terminal domain that binds the ligand (O'Hara et al., Neuron 11:41 (1993)). The V1 receptor ligand-binding pocket is formed from the transmembrane segments or by the peptide loops between the transmembrane segments. The different structure of the V1 and V2 receptor ligand binding pockets suggests these receptors recognize different types of ligands. Recent work of Krieger et al., J. Biol. Chem. 274:4655 (1999), has provided experimental evidence in support of V1 receptors being activated by lipophilic volatile ordorants, and V2 receptors interacting with proteinaceous pheromone components such as the MUPs or other lipocalins. In this way, the dual recognition of a lipocalin and its phermone ligand may be mediated separately by two distinct classes of pheromone receptors. Thus, the pheromone response is apparently due to the collective signals from these two receptor classes.

Pheromone activities affecting sexual and other behavior or development have been reported in primates. Short-chain fatty acids found in vaginal secretions of rhesus monkeys can act as sex-attractants (Keveme and Michael, J. Endocrinol. 51:313 (1971)). Estradiol was reported as a pheromonal attractant of male rhesus monkeys (Michael et al., Nature 218:746 (1968)). The removal of the vomeronasal organ in lower primates was to shown to impair male sexual behavior consistent with the existence of pheromones whose actions are mediated through the vomeronasal organ (Aujard, Physiol. Behav. 62:1003 (1997)). From these findings, it would seem that sexual behavior in primates is at least in part influenced by pheromones.

The existence of human pheromones, however, is controversial. The existence of human pheromones was first suggested by the observation that women living together can develop synchronized menstrual cycles under specific conditions (McClintock, Nature 291:244 (1971)). The causal agents were later attributed to odorless pheromone-like substances produced in female underarms (Stern and McClintock, Nature 392:177 (1998)). There are also reports suggesting short-chain fatty acids found in vaginal secretions isolated from vaginal secretion of sexually active human females can act as sex-attractants (Michael et al., Psychoneuroendocrinology 1: 153 (1975); Sokolov et al., Archives of Sexual Behavior 5:269 (1976)).

One human pheromone activity under investigation is the nipple search pheromone. Suckling is a behavior that is universal and characteristic of mammals, and the survival of every newborn is dependent on its ability to find the mother's nipples and suckle (Blass and Teicher, Science 210:15 (1980)). It is believed that the newborn is directed to the nipple by a pheromone produced by the nipple or by the surrounding areola region of the breast. This pheromone activity was first studied in non-human mammals. Rabbit, rat, and pig nipple washings were shown to contain this pheromone activity (Blass and Teicher, Science 210:15 (1980); Keil et al., Physiol. Behav. 47:525 (1990); Morrow-Tesch and McGlone, J. Anim. Sci. 68:3563 (1990)). Rabbit pups are particularly receptive to the effect of nipple-search pheromone (Hudson, Dev. Psychobiol. 18:575 (1985); Distel and Hudson, J. Comp. Physiol. A 157:599 (1985); Keil et al., Physiol. Behav. 47:525 (1990)). Although blind at birth, rabbit pups are able to locate a nipple within a few seconds after the mother's arrival. The production of rabbit nipple-search pheromone appeared to be stimulated by ovarian steroids and prolactin, and can be found in milk (Keil et al., Physiol. Behav. 47:525 (1990); Gonzalez-Mariscal et al., Biology of Reproduction 50:373 (1994)). It appears that the action of the nipple search pheromone is one of the few that is mediated by the main olfactory epithelium. Ablation of the rat, mouse or rabbit vomeronasal organ apparently has no effect on the responsiveness to the maternal nipple search pheromone (Bean et al., In Mammalian Olfaction, Reproductive Process and Behavior, Doty (Ed.), pages 143-160 (Academic Press 1976); Teicher et al., Dev. Brain Res. 12:97 (1984); Hudson and Distel, Physiol. Behav. 37:123 (1986)). In contrast, responsiveness to the pheromone was only abolished with disruption of the main olfactory epithelium (Distel and Hudson, J. Comp. Physiol A 157:599 (1985); Kovach and Kling, Anim. Behav. 15:91 (1967); McClelland and Cowley, Physiol. Behav. 9:319 (1982); Singh and Tucker, Physiol. Behav. 17:373 (1976); Teicher et al., Physiol. Behav. 21:553 (1978)). It has been suggested that as a consequence of this pheromone interacting through the main olfactory epithelium, which has connections to cognitive centers of the brain, a newborn can sometimes be reconditioned to respond to other odors in place of the nipple search pheromone (Kindermann et al, Physiol. Behav. 50:457 (1991); Fillion and Blass, Science 231:729 (1986)).

Human infants are particularly responsive to olfactory signals from their mother's breast. Almost immediately after birth, maternal breast odors elicit specific facial orientation followed by increased motor activity and arousal leading to the successful localization of the nipple and the initiation of suckling by the infant (Porter and Winberg, Neurosci. Biobehav. Rev. 23:439 (1999); Winberg and Porter, Acta Paediatr. 87:6 (1989)). The role of these maternal olfactory signals in early infant breast-feeding is functionally analogous to the nipple-search pheromone described in rodents, pigs and rabbits. The nipple and areola region is supplied with a dense accumulation of skin glands that could be the source of the attractive signal. In particular, ducts of the sebaceous glands open directly on the tip of the nipple and are enlarged during lactation. In addition to helping to guide the infant directly to the nipple, maternal breast odors also affect a number of other neonatal behavior that increase the probability of successful nipple grasping and feeding. Collectively, these early olfactory-based recognition events are important factors in the development of the infant-mother bond.

Much of the human pheromone research has centered on the 16-androstenes, which comprise a family of related steroids that have pheromone activity in animals. Androsterone (5-α-16-androst-16-en-3-one) and its alcohol form, androstenol (5-α-16-androst-16-en-3-ol) are porcine pheromones synthesized in the boar testes and submaxillary glands and, which induce recipient sows to adopt the mating stance (Reed and Melrose, Br. Vet. J. 130:61 (1974); Perry et al., Animal Production 31:191 (1980)). These and other related 16-androstenes are also synthesized in human testes and believed by many investigators to have pheromone activity in humans (see, for example, Gower and Ruparelia, J. Endocrinol. 137:167 (1993); U.S. Pat. No. 5,278,241; U.S. Pat. No. 5,272,134; U.S. Pat. No. 5,969,168; U.S. Pat. No. 5,965,552). 5-α-16-androst-16-en-3-ol is the most abundant of the 16-androstenes in human urine. Androsta-4,16-dien-3-one is the most abundant 16-androstene present in human semen, in male axillary hair and male axillary skin surfaces (Nixon et al., J. Steroid Biochem. Mol. Biol. 29:505 (1988); Rennie et al., In: Chemical Signals in Vertebrates, pages 55-60 (Oxford University Press 1990); Kwan et al., J. Steroid Biochem. Mol. Biol. 43:549 (1992)). Androstenes are also found in the human axillary sweat secreted by the apocrine glands, which are sites for pheromone production in lower animals (Brooksbank et al., Experientia 30:864 (1994)).

Androsta-4,16,-dien-3-one was reported to stimulate the human vomeronasal organ (Jennings-White, Perfum. Flav. 20:1 (1995); Monti-Bloch et al., Chem. Sens. 23:114 (1998)). The administration of androstadienone at picogram levels directly to the human female vomeronasal organ was found to reduce discomfort and tension (Grosser et al., Psychoneuroendocrinology 25:289 (2000)). While other studies also suggested that 16-androstenes and other putative pheromones could indeed alter human social behaviors, there are also reports of negative and contradictory results (Filsinger et al., J. Comp. Psychol. 98:219 (1984); Gustavson et al., Psychol. 101:210 (1987); Cowley and Brooksbank, J. Steroid Biochem. Molec. Biol. 39:647 (1991); Gower and Ruparelia, J. Endocrinol. 137:167 (1993); Pause et al., Physiology & Behavior 68:129 (1999)). The inconsistent findings have been attributed to different forms or formulations of the 16-androstenes used, or due to the subjectivity and difficulties associated with human behavioral studies.

An alternative explanation is that a more robust reproducible human pheromone response to the androstrenes or to other potential small chemical pheromones such as the estrenes (U.S. Pat. Nos. 5,272,134, 5,278,141, and 5,994,568) may require a human lipocalin carrier protein. Such a lipocalin carrier protein may alone have phermone activity, or may augment the activity of its phermone ligand. In addition to hamster aphrodisin and the rodent MUPs, there have only been a few examples of characterized proteins associated with pheromones in mammals and none so far in humans. Booth and White reported a partially characterized porcine extracellular protein, pheromaxein, that binds androstenol and related steroids in boar submaxillary gland saliva (Booth and White, J. Endrocr. 118:47 (1988)). A salivary gland lipocalin, which binds 16-androstrenes, was later isolated from boar submaxillary gland (Marchese et al., Eur. J. Biochem. 252:563 (1998)). A cDNA encoding this protein, termed sex-specific salivary lipocalin (SAL), was recently reported, and shown to encode a polypeptide with high homology to the murine MUPs (Loebel et al., Biochem. J. 350:369 (2000)). It is not known whether boar SAL or pheromaxein has phermone activity itself, or may contribute to the phermone activity of cognate androstene ligands. Neither human homologs of boar pheromaxein or boar SAL have been isolated. However, a human lipocalin, apolipoprotein D, was recently found expressed in apocrine glands, and was shown to bind the axillary odorant, E-3-methyl-2-hexenoic acid (E-3M2H) (Zeng et al., Proc. Nat'l Acad. Sci. (USA) 93:6626 (1996)). E-3M2H and its isomers are major ordorants in the human axillary region. Although studies have implicated axillary odors and secretions in the alterations of menstrual cycle and mood changes, the role of E-3M2H or apolipoprotein D in these responses has not been evaluated. (McClintock, Nature 291:244 (1971); Stem and McClintock, Nature 392:177 (1998)).

Hence, there is an unfulfilled need for human pheromones and agents that can augment the pheromone response. Human lipocalin proteins that are produced in the genital tract may be used as a phermone, or to support pheromone action in the alternation of human reproductive physiology or behavior. Likewise, lipocalins that are produced in the human breast can also be used for these purposes. In addition, a breast lipocalin may be an important component of the olfactory signals between mother and infant. In particular, a lipocalin produced in breast tissues may mediate the nipple search behavior in infants.

### BRIEF SUMMARY OF THE INVENTION

The present invention describes novel uses for two human lipocalin proteins, glycodelin and Zlipo1, that are expressed in the genital tract and in the breast. Their expression in these tissues and their structural similarity to known rodent pheromones and pheromone carrier proteins indicate that these lipocalin proteins are useful in olfaction-mediated chemical communication between individuals.

### DETAILED DESCRIPTION OF THE INVENTION

1. Overview

Zlipo1, also known as hOBP11b, is a lipocalin that is produced in breast, testes, and prostate (Conklin, U.S. Pat. No. 6,020,163; Lacazette et al., Human Mol. Genet. 9:289 (2000)). Zlipo1 nucleotide and amino acid sequences are disclosed herein as SEQ ID NO:1 and SEQ ID NO:2, respectively. Glycodelin, also known as placental protein 14, is another member of the lipocalin family of proteins (Julkunen et al., Proc. Nat'l Acad. Sci. (USA) 85:8845 (1988); Genbank accession number J04129). Glycodelin appears as various glycoforms with different biological activities in the endometrium (glycodelin-A) and in seminal plasma (glycodelin-S). The precise functions of the glycodelins are not known. However, glycodelin-A displays contraceptive and immunosuppressive properties (Oehninger et al., Fertil. Steril. 63:377 (1995); Okamoto et al., Am. J. Reprod. Immunol. 26:137 (1991); Bolton et al., Lancet 1:593 (1987)). The glycodelin-S glycoform in the seminal plasma apparently does not have contraceptive activity (Koistinen et al., Lab. Invest. 76:683 (1997); Morris et al., J. Biol. Chem. 271:32159 (1996)). In the breast, glycodelin is expressed in the epithelium and in the ductal tissues. A glycodelin mRNA splicing variant that lacks exon 4 was detected in breast tissues. This mRNA variant encodes a polypeptide lacking the potential N-glycosylation site at Asn-85, which may result in a different biological activity from the glycodelins expressed in the genital tract (Kamarainen et al., Int. J. Cancer 83:738 (1999)).

The present invention contemplates methods for detecting a Zlipo1 receptor or a glycodelin receptor within a test sample, comprising the steps of (a) contacting the test sample with a polypeptide that comprises the amino acid sequence of SEQ ID NO:2 or SEQ ID NO:4, and (b) detecting the binding of the polypeptide to receptor in the sample. Such an assay can be performed with cultured cells that may express the cognate receptor, and the detecting step would comprise measuring a biological response in the cultured cell. In another variation of these methods, the source of a putative Zlipo1 receptor or glycodelin receptor is a cell membrane preparation obtained from cells that produce the receptor. In either approach, one suitable type of cell is a recombinant host cell transfected with a cDNA library prepared from vomeronasal tissue or from main olfactory epithelium tissue. One suitable source of such tissue is human tissue.

The present invention also provides methods for identifying a phermone ligand, which binds to Zlipo1 or glycodelin. In one approach, for example, the presence of a Zlipo1 ligand or a glycodelin ligand in a test sample is detected by: (a) contacting the test sample with a Zlipo1 or glycodelin polypeptide that comprises the amino acid sequence of SEQ ID NO:2 or the amino acid sequence of SEQ ID NO:4, and (b) detecting the binding of the polypeptide to ligand in the test sample.

The present invention also contemplates the isolation of Zlipo1 /glycodelin ligands and receptors.

The present invention further provides pharmaceutical compositions comprising Zlipo1 or glycodelin. These compositions may be conveniently provided in a form suitable for nasal administration.

These and other aspects of the invention will become evident upon reference to the following detailed description. In addition, various references are identified below and are incorporated by reference in their entirety.

2. Definitions

In the description that follows, a number of terms are used extensively. The following definitions are provided to facilitate understanding of the invention.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence. For example, the sequence 5′ ATGCACGGG 3′ is complementary to 5′ CCCGTGCAT 3′.

The term “contig” denotes a nucleic acid molecule that has a contiguous stretch of identical or complementary sequence to another nucleic acid molecule. Contiguous sequences are said to “overlap” a given stretch of a nucleic acid molecule either in their entirety or along a partial stretch of the nucleic acid molecule.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SPI, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

An “integrated genetic element” is a segment of DNA that has been incorporated into a chromosome of a host cell after that element is introduced into the cell through human manipulation. Within the present invention, integrated genetic elements are most commonly derived from linearized plasmids that are introduced into the cells by electroporation or other techniques. Integrated genetic elements are passed from the original host cell to its progeny.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces Zlipo1 from an expression vector. In contrast, Zlipo1 can be produced by a cell that is a “natural source” of Zlipo1, and that lacks an expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

The term “secretory signal sequence” denotes a nucleotide sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “anti-idiotype antibody” is an antibody that binds with the variable region domain of an immunoglobulin. In the present context, an anti-idiotype antibody binds with the variable region of an anti-Zlipo1 antibody, and thus, an anti-idiotype antibody mimics an epitope of Zlipo1 .

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-glycodelin monoclonal antibody fragment binds with an epitope of glycodelin.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). Nucleic acid molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

An “anti-sense oligonucleotide specific for Zlipo1” or a “Zlipo1 anti-sense oligonucleotide” is an oligonucleotide having a sequence (a) capable of forming a stable triplex with a portion of the Zlipo1 gene, or (b) capable of forming a stable duplex with a portion of an mRNA transcript of the Zlipo1 gene. Similarly, an “anti-sense oligonucleotide specific for glycodelin” or a “glycodelin anti-sense oligonucleotide” is an oligonucleotide having a sequence (a) capable of forming a stable triplex with a portion of the glycodelin gene, or (b) capable of forming a stable duplex with a portion of an mRNA transcript of the glycodelin gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

Due to the imprecision of standard analytical methods, molecular weights and lengths of polymers are understood to be approximate values. When such a value is expressed as “about” X or “approximately” X, the stated value of X will be understood to be accurate to ±10%.

3. Production of Nucleic Acid Molecules Encoding Zlipo1 and Glycodelin

Nucleic acid molecules encoding human Zlipo1 or glycodelin can be obtained by screening a human cDNA or genomic library using polynucleotide probes based upon SEQ ID NOs:1 and 3. These techniques are standard and well-established (see, for example, Ausubel et al. (eds.), Short Protocols in Molecular Biology, 3^{rd} Edition, pages 4-1 to 4-6 (John Wiley & Sons 1995) [“Ausubel (1995)”]; Wu et al., Methods in Gene Biotechnology, pages 33-41 (CRC Press, Inc. 1997) [“Wu (1997)”]).

As an alternative, a nucleic acid molecule encoding human Zlipo1 or glycodelin can be obtained by synthesizing nucleic acid molecules using mutually priming long oligonucleotides and the nucleotide sequences described herein (see, for example, Ausubel (1995) at pages 8-8 to 8-9). Established techniques using the polymerase chain reaction provide the ability to synthesize DNA molecules at least two kilobases in length (Adang et al., Plant Molec. Biol. 21:1131 (1993), Bambot et al., PCR Methods and Applications 2:266 (1993), Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993), and Holowachuk et al., PCR Methods Appl. 4:299 (1995)).

Nucleic acid molecules, encoding Zlipo1 or glycodelin, can also be synthesized with “gene machines” using protocols such as the phosphoramidite method. If chemically-synthesized double stranded DNA is required for an application such as the synthesis of a gene or a gene fragment, then each complementary strand is made separately. The production of short genes (60 to 80 base pairs) is technically straightforward and can be accomplished by synthesizing the complementary strands and then annealing them. For the production of longer genes (>300 base pairs), however, special strategies may be required, because the coupling efficiency of each cycle during chemical DNA synthesis is seldom 100%. To overcome this problem, synthetic genes (double-stranded) are assembled in modular form from single-stranded fragments that are from 20 to 100 nucleotides in length. For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633 (1990).

The present invention also contemplates the use of nucleic acid molecules that encodes variants of the Zlipo1 and glycodelin polypeptides described herein. For example, those skilled in the art will recognize that the sequences disclosed herein represent single alleles of human Zlipo1 and glycodelin, and that allelic variation and alternative splicing are expected to occur. Allelic variants of this sequence can be cloned by probing cDNA or genomic libraries from different individuals according to standard procedures. Allelic variants and splice variants of these sequences can be cloned by probing cDNA or genomic libraries from different individuals or tissues according to standard procedures known in the art.

The present invention also contemplates the use of Zlipo1 and glycodelin that have a substantially similar sequence identity to the polypeptides of SEQ ID NOs:2 and 4, or orthologs. The term “substantially similar sequence identity” is used herein to denote polypeptides having 70%, 80%, 90%, 95%, 96%, 97%, 98%, or 99% sequence identity to the sequence shown in SEQ ID NOs:2 and 4.

Percent sequence identity is determined by conventional methods. See, for example, Altschul et al., Bull. Math. Bio. 48:603 (1986), and Henikoff and Henikoff, Proc. Nat'l Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 3 (amino acids are indicated by the standard one-letter codes). The percent identity is then calculated as: ([Total number of identical matches]/[length of the longer sequence plus the number of gaps introduced into the longer sequence in order to align the two sequences])(100).

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative Zlipo1/glycodelin variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO:2 or SEQ ID NO:4) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

The present invention includes the use of polypeptides having a conservative amino acid change, compared with the amino acid sequence of SEQ ID NOs:2 and 4. That is, variants can be obtained that contain one or more amino acid substitutions of SEQ ID NOs:2 and 4, in which an alkyl amino acid is substituted for an alkyl amino acid in a Zlipo1 or glycodelin amino acid sequence, an aromatic amino acid is substituted for an aromatic amino acid in a Zlipo1 or glycodelin amino acid sequence, a sulfur-containing amino acid is substituted for a sulfur-containing amino acid in a Zlipo1 or glycodelin amino acid sequence, a hydroxy-containing amino acid is substituted for a hydroxy-containing amino acid in a Zlipo1 or glycodelin amino acid sequence, an acidic amino acid is substituted for an acidic amino acid in a Zlipo1 or glycodelin amino acid sequence, a basic amino acid is substituted for a basic amino acid in a Zlipo1 or glycodelin amino acid sequence, or a dibasic monocarboxylic amino acid is substituted for a dibasic monocarboxylic amino acid in a Zlipo1 or glycodelin amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine.

Particular variants of Zlipo1 or glycodelin are characterized by having greater than 96%, at least 97%, at least 98%, or at least 99% sequence identity to the corresponding amino acid sequence, wherein the variation in amino acid sequence is due to one or more conservative amino acid substitutions.

Conservative amino acid changes in a Zlipo1 or glycodelin gene can be introduced by substituting nucleotides for the nucleotides recited in SEQ ID NOs:1 and 3. Such “conservative amino acid” variants can be obtained, for example, by oligonucleotide-directed mutagenesis, linker-scanning mutagenesis, mutagenesis using the polymerase chain reaction, and the like (see Ausubel (1995) at pages 8-10 to 8-22; and McPherson (ed.), Directed Mutagenesis: A Practical Approach (IRL Press 1991)).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is typically carried out in a cell-free system comprising an E. coli S30 extract and commercially available enzymes and other reagents. Proteins are purified by chromatography. See, for example, Robertson et al., J. Am. Chem. Soc. 113:2722 (1991), Ellman et al., Methods Enzymol. 202:301 (1991), Chung et al., Science 259:806 (1993), and Chung et al., Proc. Nat'l Acad. Sci. USA 90:10145 (1993).

In a second method, translation is carried out in Xenopus oocytes by microinjection of mutated mRNA and chemically aminoacylated suppressor tRNAs (Turcatti et al., J. Biol. Chem. 271:19991 (1996)). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470 (1994). Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395 (1993)).

A limited number of non-conservative amino acids, amino acids that are not encoded by the genetic code, non-naturally occurring amino acids, and unnatural amino acids may be substituted for Zlipo1 or glycodelin amino acid residues.

Multiple amino acid substitutions can be made and tested using known methods of mutagenesis and screening, such as those disclosed by Reidhaar-Olson and Sauer (Science 241:53 (1988)) or Bowie and Sauer (Proc. Nat'l Acad. Sci. USA 86:2152 (1989)). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832 (1991), Ladner et al., U.S. Pat. No. 5,223,409, Huse, international publication No. WO 92/06204, and region-directed mutagenesis (Derbyshire et al., Gene 46:145 (1986), and Ner et al., DNA 7:127, (1988)).

Variants of the disclosed Zlipo1 or glycodelin nucleotide and polypeptide sequences can also be generated through DNA shuffling as disclosed by Stemmer, Nature 370:389 (1994), Stemmer, Proc. Nat'l Acad. Sci. USA 91:10747 (1994), and international publication No. WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

The present invention also includes the use of “functional fragments” of Zlipo1 or glycodelin polypeptides and nucleic acid molecules encoding such functional fragments. Routine deletion analyses of nucleic acid molecules can be performed to obtain functional fragments of a nucleic acid molecule that encodes a Zlipo1 or glycodelin polypeptide. As an illustration, DNA molecules having the nucleotide sequence of SEQ ID NO:1 can be digested with Bal31 nuclease to obtain a series of nested deletions. One alternative to exonuclease digestion is to use oligonucleotide-directed mutagenesis to introduce deletions or stop codons to specify production of a desired fragment.

As an illustration, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993), Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987), Herschman, “The EGF Receptor,” in Control of Animal Cell Proliferation, Vol. 1, Boynton et al., (eds.) pages 169-199 (Academic Press 1985), Coumailleau et al., J. Biol. Chem. 270:29270 (1995); Fukunaga et al., J. Biol. Chem. 270:25291 (1995); Yamaguchi et al., Biochem. Pharmacol. 50:1295 (1995), and Meisel et al., Plant Molec. Biol. 30:1 (1996).

4. Production of Fusion Proteins

Fusion proteins of Zlipo1 or glycodelin can be used to produce the polypeptides in a recombinant host, and to isolate the polypeptides. One type of fusion protein comprises a peptide that guides a Zlipo1 or glycodelin polypeptide from a recombinant host cell. To direct a Zlipo1 or glycodelin polypeptide into the secretory pathway of a eukaryotic host cell, a secretory signal sequence (also known as a signal peptide, a leader sequence, prepro sequence or pre sequence) is provided in the expression vector. While the secretory signal sequence may be derived from Zlipo1 or glycodelin, a suitable signal sequence may also be derived from another secreted protein or synthesized de novo. The secretory signal sequence is operably linked to a Zlipo1- or glycodelin-encoding sequence such that the two sequences are joined in the correct reading frame and positioned to direct the newly synthesized polypeptide into the secretory pathway of the host cell. Secretory signal sequences are commonly positioned 5′ to the nucleotide sequence encoding the polypeptide of interest, although certain secretory signal sequences may be positioned elsewhere in the nucleotide sequence of interest (see, e.g., Welch et al., U.S. Pat. No. 5,037,743; Holland et al., U.S. Pat. No. 5,143,830).

While the secretory signal sequence of Zlipo1 or glycodelin, or another protein produced by mammalian cells (e.g., tissue-type plasminogen activator signal sequence, as described, for example, in U.S. Pat. No. 5,641,655) is useful for expression of Zlipo1 or glycodelin in recombinant mammalian hosts, a yeast signal sequence is preferred for expression in yeast cells. Examples of suitable yeast signal sequences are those derived from yeast mating phermone α-factor (encoded by the MFα1 gene), invertase (encoded by the SUC2 gene), or acid phosphatase (encoded by the PHO5 gene). See, for example, Romanos et al., “Expression of Cloned Genes in Yeast,” in DNA Cloning 2: A Practical Approach, 2^{nd} Edition, Glover and Hames (eds.), pages 123-167 (Oxford University Press 1995).

In bacterial cells, it is often desirable to express a heterologous protein as a fusion protein to decrease toxicity, increase stability, and to enhance recovery of the expressed protein. For example, Zlipo1 or glycodelin can be expressed as a fusion protein comprising a glutathione S-transferase polypeptide. Glutathione S-transferease fusion proteins are typically soluble, and easily purifiable from E. coli lysates on immobilized glutathione columns. In similar approaches, a Zlipo1 or glycodelin fusion protein comprising a maltose binding protein polypeptide can be isolated with an amylose resin column, while a fusion protein comprising the C-terminal end of a truncated Protein A gene can be purified using IgG-Sepharose. Established techniques for expressing a heterologous polypeptide as a fusion protein in a bacterial cell are described, for example, by Williams et al., “Expression of Foreign Proteins in E. coli Using Plasmid Vectors and Purification of Specific Polyclonal Antibodies,” in DNA Cloning 2: A Practical Approach, 2^{nd} Edition, Glover and Hames (Eds.), pages 15-58 (Oxford University Press 1995). In addition, commercially available expression systems are available. For example, the PINPOINT Xa protein purification system (Promega Corporation; Madison, Wis.) provides a method for isolating a fusion protein comprising a polypeptide that becomes biotinylated during expression with a resin that comprises avidin.

Peptide tags that are useful for isolating heterologous polypeptides expressed by either prokaryotic or eukaryotic cells include polyHistidine tags (which have an affinity for nickel-chelating resin), c-myc tags, calmodulin binding protein (isolated with calmodulin affinity chromatography), substance P, the RYIRS tag (which binds with anti-RYIRS antibodies), the Glu-Glu tag, and the FLAG tag (which binds with anti-FLAG antibodies). See, for example, Luo et al., Arch. Biochem. Biophys. 329:215 (1996), Morganti et al., Biotechnol. Appl. Biochem. 23:67 (1996), and Zheng et al., Gene 186:55 (1997). Nucleic acid molecules encoding such peptide tags are available, for example, from Sigma-Aldrich Corporation (St. Louis, Mo.).

Another form of fusion protein comprises a Zlipo1 or glycodelin polypeptide and an immunoglobulin heavy chain constant region, typically an F_{c} fragment, which contains two constant region domains and a hinge region but lacks the variable region. As an illustration, Chang et al., U.S. Pat. No. 5,723,125, describe a fusion protein comprising a human interferon and a human immunoglobulin Fc fragment, in which the C-terminal of the interferon is linked to the N-terminal of the Fc fragment by a peptide linker moiety. An example of a peptide linker is a peptide comprising primarily a T cell inert sequence, which is immunologically inert. An exemplary peptide linker has the amino acid sequence: GGSGG SGGGG SGGGG S (SEQ ID NO:5). In such a fusion protein, an illustrative Fc moiety is a human γ4 chain, which is stable in solution and has little or no complement activating activity. Accordingly, the present invention contemplates the use of a Zlipo1 or glycodelin fusion protein that comprises a Zlipo1 or glycodelin moiety and a human Fc fragment, wherein the C-terminus of the Zlipo1 or glycodelin moiety is attached to the N-terminus of the Fc fragment via a peptide linker, such as a peptide consisting of the amino acid sequence of SEQ ID NO:5. The Zlipo1 or glycodelin moiety can be a Zlipo1 or glycodelin molecule, or a fragment thereof.

In another variation, a Zlipo1 or glycodelin fusion protein comprises an IgG sequence, a Zlipo1 or glycodelin moiety covalently joined to the aminoterminal end of the IgG sequence, and a signal peptide that is covalently joined to the aminoterminal of the Zlipo1 or glycodelin moiety, wherein the IgG sequence consists of the following elements in the following order: a hinge region, a CH₂ domain, and a CH₃ domain. Accordingly, the IgG sequence lacks a CH₁ domain. The Zlipo1 or glycodelin moiety displays a Zlipo1 or glycodelin activity, as described herein, such as the ability to bind with a Zlipo1 or glycodelin antibody. This general approach to producing fusion proteins that comprise both antibody and nonantibody portions has been described by LaRochelle et al., EP 742830 (WO 95/21258).

Fusion proteins comprising a Zlipo1 or glycodelin moiety and an Fc moiety can be used, for example, as an in vitro assay tool. For example, the presence of a Zlipo1 or glycodelin receptor in a biological sample can be detected using a Zlipo1- or glycodelin-antibody fusion protein, in which the Zlipo1 or glycodelin moiety is used to target the cognate receptor, and a macromolecule, such as Protein A or anti-Fc antibody, is used to detect the bound fusion protein-receptor complex. Furthermore, such fusion proteins can be used to identify agonists and antagonists that interfere with the binding of Zlipo1 or glycodelin to its receptor.

Fusion proteins can be prepared by methods known to those skilled in the art by preparing each component of the fusion protein and chemically conjugating the components. Alternatively, a polynucleotide encoding both components of the fusion protein in the proper reading frame can be generated using known techniques and expressed by the methods described herein. General methods for enzymatic and chemical cleavage of fusion proteins are described, for example, by Ausubel (1995) at pages 16-19 to 16-25.

5. Production of Polypeptides

The polypeptides of the present invention, including full-length polypeptides, functional fragments, and fusion proteins, can be produced in recombinant host cells following conventional techniques. To express a Zlipo1 or glycodelin gene, a nucleic acid molecule encoding the polypeptide must be operably linked to regulatory sequences that control transcriptional expression in an expression vector and then, introduced into a host cell. In addition to transcriptional regulatory sequences, such as promoters and enhancers, expression vectors can include translational regulatory sequences and a marker gene, which is suitable for selection of cells that carry the expression vector.

Zlipo1 or glycodelin polypeptides can be expressed in any prokaryotic or eukaryotic host cell. Preferably, the polypeptides are produced by a eukaryotic cell, such as a mammalian cell, fungal cell, insect cell, avian cell, and the like. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44 (Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658).

A nucleic acid molecules encoding a Zlipo1 or glycodelin polypeptide can be introduced into host cells using a variety of standard techniques including calcium phosphate transfection, liposome-mediated transfection, microprojectile-mediated delivery, electroporation, and the like. Transfected cells can be selected and propagated to provide recombinant host cells that comprise the gene of interest stably integrated in the host cell genome.

The baculovirus system provides an efficient means to introduce cloned genes of interest into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. A second method of making recombinant baculovirus utilizes a transposon-based system described by Luckow (Luckow, et al., J. Virol. 67:4566 (1993)). This system, which utilizes transfer vectors, is sold in the BAC-to-BAC kit (Life Technologies, Rockville, Md.). This system utilizes a transfer vector, PFASTBAC (Life Technologies) containing a Tn7 transposon to move the DNA encoding the a Zlipo1 or glycodelin polypeptide into a baculovirus genome maintained in E. coli as a large plasmid called a “bacmid.” See, Hill-Perkins and Possee, J. Gen. Virol. 71:971 (1990), Bonning, et al., J. Gen. Virol. 75:1551 (1994), and Chazenbalk, and Rapoport, J. Biol. Chem. 270:1543 (1995). In addition, transfer vectors can include an in-frame fusion with DNA encoding an epitope tag at the C- or N-terminus of the expressed polypeptide, for example, a Glu-Glu epitope tag (Grussenmeyer et al., Proc. Nat'l Acad. Sci. 82:7952 (1985)). Using a technique known in the art, a transfer vector containing a gene of interest is transformed into E. coli, and screened for bacmids, which contain an interrupted lacZ gene indicative of recombinant baculovirus. The bacmid DNA containing the recombinant baculovirus genome is then isolated using common techniques.

The recombinant virus or bacmid is used to transfect host cells. Suitable insect host cells include cell lines derived from IPLB-Sf-21, a Spodoptera frugiperda pupal ovarian cell line, such as Sƒ9 (ATCC CRL 1711), Sƒ21AE, and Sƒ21 (Invitrogen Corporation; San Diego, Calif.), as well as Drosophila Schneider-2 cells, and the HIGH FIVEO cell line (Invitrogen) derived from Trichoplusia ni (U.S. Pat. No. 5,300,435). Commercially available serum-free media can be used to grow and to maintain the cells. Suitable media are Sf900 II™ (Life Technologies) or ESF 921™ (Expression Systems) for the Sf9 cells; and Ex-cellO405™ (JRH Biosciences, Lenexa, Kan.) or Express FiveO™ (Life Technologies) for the T. ni cells. When recombinant virus is used, the cells are typically grown up from an inoculation density of approximately 2-5×10⁵ cells to a density of 1-2×10⁶ cells at which time a recombinant viral stock is added at a multiplicity of infection of 0.1 to 10, more typically near 3.

Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

Fungal cells, including yeast cells, can also be used to produce a Zlipo1 or glycodelin polypeptide. Yeast species of particular interest in this regard include Saccharomyces cerevisiae, Pichia pastoris, and Pichia methanolica. Suitable promoters for expression in yeast include promoters from GAL1 (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. Methods for transforming S. cerevisiae cells with exogenous DNA and producing recombinant polypeptides therefrom are disclosed by, for example, Kawasaki, U.S. Pat. No. 4,599,311, Kawasaki et al., U.S. Pat. No. 4,931,373, Brake, U.S. Pat. No. 4,870,008, Welch et al., U.S. Pat. No. 5,037,743, and Murray et al., U.S. Pat. No. 4,845,075. Transformed cells are selected by phenotype determined by the selectable marker, commonly drug resistance or the ability to grow in the absence of a particular nutrient (e.g., leucine). An illustrative vector system for use in Saccharomyces cerevisiae is the POT1 vector system disclosed by Kawasaki et al. (U.S. Pat. No. 4,931,373), which allows transformed cells to be selected by growth in glucose-containing media. Additional suitable promoters and terminators for use in yeast include those from glycolytic enzyme genes (see, e.g., Kawasaki, U.S. Pat. No. 4,599,311, Kingsman et al., U.S. Pat. No. 4,615,974, and Bitter, U.S. Pat. No. 4,977,092) and alcohol dehydrogenase genes. See also U.S. Pat. Nos. 4,990,446, 5,063,154, 5,139,936, and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillennondii and Candida maltosa are known in the art. See, for example, Gleeson et al., J. Gen. Microbiol. 132:3459 (1986), and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

For example, the use of Pichia methanolica as host for the production of recombinant proteins is disclosed by Raymond, U.S. Pat. No. 5,716,808, Raymond, U.S. Pat. No. 5,736,383, Raymond et al., Yeast 14:11-23 (1998), and in international publication Nos. WO 97/17450, WO 97/17451, WO 98/02536, and WO 98/02565. DNA molecules for use in transforming P. methanolica will commonly be prepared as double-stranded, circular plasmids, which can be linearized prior to transformation. For polypeptide production in P. methanolica, the promoter and terminator in the plasmid can be that of a P. methanolica gene, such as a P. methanolica alcohol utilization gene (AUG1 or AUG2). Other useful promoters include those of the dihydroxyacetone synthase, formate dehydrogenase, and catalase genes. To facilitate integration of the DNA into the host chromosome, the entire expression segment of the plasmid can be flanked at both ends by host DNA sequences. For large-scale, industrial processes where it is desirable to minimize the use of methanol host cells can be used in which both methanol utilization genes (AUG1 and AUG2) are deleted. For production of secreted proteins, host cells can be used that are deficient in vacuolar protease genes (PEP4 and PRB1). Electroporation is used to facilitate the introduction of a plasmid containing DNA encoding a polypeptide of interest into P. methanolica cells. P. methanolica cells can be transformed by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Nucleic acid molecules encoding a Zlipo1 or glycodelin polypeptide can also be introduced into plant protoplasts, intact plant tissues, or isolated plant cells. Methods for introducing nucleic acid molecules into plant tissue include the direct infection or co-cultivation of plant tissue with Agrobacterium tumefaciens, microprojectile-mediated delivery, DNA injection, electroporation, and the like. See, for example, Horsch et al., Science 227:1229 (1985), Klein et al., Biotechnology 10:268 (1992), and Miki et al., “Procedures for Introducing Foreign DNA into Plants,” in Methods in Plant Molecular Biology and Biotechnology, Glick et al. (eds.), pages 67-88 (CRC Press, 1993).

Standard methods for introducing nucleic acid molecules into bacterial, yeast, insect, mammalian, and plant cells are provided, for example, by Ausubel (1995). General methods for expressing and recovering foreign protein produced by a mammalian cell system are provided by, for example, Etcheverry, “Expression of Engineered Proteins in Mammalian Cell Culture,” in Protein Engineering: Principles and Practice, Cleland et al. (eds.), pages 163 (Wiley-Liss, Inc. 1996). Established methods for isolating recombinant proteins from a baculovirus system are described by Richardson (ed.), Baculovirus Expression Protocols (The Humana Press, Inc. 1995).

As an alternative, polypeptides described herein can be synthesized by exclusive solid phase synthesis, partial solid phase methods, fragment condensation or classical solution synthesis. These synthesis methods are well-known to those of skill in the art (see, for example, Merrifield, J. Am. Chem. Soc. 85:2149 (1963), Stewart et al., “Solid Phase Peptide Synthesis” (2nd Edition), (Pierce Chemical Co. 1984), Bayer and Rapp, Chem. Pept. Prot. 3:3 (1986), Atherton et al., Solid Phase Peptide Synthesis: A Practical Approach (IRL Press 1989), Fields and Colowick, “Solid-Phase Peptide Synthesis,” Methods in Enzymology Volume 289 (Academic Press 1997), and Lloyd-Williams et al., Chemical Approaches to the Synthesis of Peptides and Proteins (CRC Press, Inc. 1997)). Variations in total chemical synthesis strategies, such as “native chemical ligation” and “expressed protein ligation” are also standard (see, for example, Dawson et al., Science 266:776 (1994), Hackeng et al., Proc. Nat'l Acad. Sci. USA 94:7845 (1997), Dawson, Methods Enzymol. 287: 34 (1997), Muir et al, Proc. Nat'l Acad. Sci. USA 95:6705 (1998), Severinov and Muir, J. Biol. Chem. 273:16205 (1998),

Zlipo1 or glycodelin polypeptides can be purified to at least about 80% purity, to at least about 90% purity, to at least about 95% purity, or greater than 95% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. Certain purified polypeptide preparations are substantially free of other polypeptides, particularly other polypeptides of animal origin.

Fractionation and/or conventional purification methods can be used to obtain preparations of Zlipo1 or glycodelin polypeptides purified from natural sources, and recombinant polypeptides and fusion proteins purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. See, for example, Affinity Chromatography: Principles & Methods (Pharmacia LKB Biotechnology 1988), and Doonan, Protein Purification Protocols (The Humana Press 1996).

Zlipo1 or glycodelin polypeptides can also be isolated by exploitation of particular properties. For example, immobilized metal ion adsorption chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher, (ed.), Meth. Enzymol. 182:529 (1990)). Within additional embodiments of the invention, a fusion of the polypeptide of interest and an affinity tag (e.g., maltose-binding protein, an immunoglobulin domain) may be constructed to facilitate purification.

6. Zlipo1 and Glycodelin Analogs, Receptors, and Ligands

One general class of analogs comprises variants of Zlipo1 or glycodelin, which have an amino acid sequence that is a mutation of the amino acid sequence disclosed herein. Another general class of analogs is provided by anti-idiotype antibodies, and fragments thereof. Moreover, recombinant antibodies comprising anti-idiotype variable domains can be used as analogs (see, for example, Monfardini et al., Proc. Assoc. Am. Physicians 108:420 (1996)). Since the variable domains of anti-idiotype Zlipo1 or glycodelin antibodies mimic Zlipo1 or glycodelin, these domains can provide either agonist or antagonist activity. As an illustration, Lim and Langer, J. Interferon Res. 13:295 (1993), describe anti-idiotypic interferon-α antibodies that have the properties of either interferon-α agonists or antagonists.

Another approach to identifying Zlipo1 or glycodelin analogs is provided by the use of combinatorial libraries. Methods for constructing and screening phage display and other combinatorial libraries are provided, for example, by Kay et al., Phage Display of Peptides and Proteins (Academic Press 1996), Verdine, U.S. Pat. No. 5,783,384, Kay, et. al., U.S. Pat. No. 5,747,334, and Kauffman et al., U.S. Pat. No. 5,723,323.

Zlipo1 or glycodelin polypeptides can be used to identify small molecules that bind Zlipo1 or glycodelin (“a Zlipo1 ligand” or “a glycodelin ligand”), as well as proteins that bind with Zlipo1 or glycodelin (“a Zlipo1 receptor” or “a glycodelin receptor”). For example, Zlipo1 or glycodelin ligands can be identified by determining whether potential ligands bind with the polypeptides in vitro. In these assays, either the putative ligand or the lipocalin (Zlipo1 or glycodelin) may be detectably labeled. Methods for detecting a ligand be performed in solution or using a Zlipo1 or glycodelin polypeptide attached to a solid support. General methods for performing binding assays are well known to those of skill in the art.

Anti-idiotype Zlipo1/glycodelin antibodies, as well as Zlipo1/glycodelin polypeptides can be used to identify and to isolate cognate receptors. For example, Zlipo1 or glycodelin proteins and peptides can be immobilized on a column and used to bind receptor proteins from membrane preparations that are run over the column (Hermanson et al. (eds.), Immobilized Affinity Ligand Techniques, pages 195-202 (Academic Press 1992)). Also see, Varthakavi and Minocha, J. Gen. Virol. 77:1875 (1996), who describe the use of anti-idiotype antibodies for receptor identification. In another approach, receptor proteins that bind Zlipo1/glycodelin can isolated from cell membranes by photocrosslinking, solubilizing, and then immunoprecipitating complexes of Zlipo1/glycodelin and the cognate receptor using antibodies to Zlipo1/glycodelin.

Radiolabeled or affinity labeled Zlipo1/glycodelin polypeptides can also be used to identify or to localize cognate receptors in a biological sample (see, for example, Deutscher (ed.), Methods in Enzymol., 182:721-37 (Academic Press 1990); Brunner et al., Ann. Rev. Biochem. 62:483 (1993); Fedan et al., Biochem. Pharmacol. 33:1167 (1984)). Moreover, Zlipo1/glycodelin labeled with biotin or FITC can be used for expression cloning of receptors. Alternatively, a cDNA encoding a Zlipo1/glycodelin receptor can be isolated from a vomeronasal organ cDNA library, or a cDNA library produced from main olfactory epithelium, by expression cloning protocols similar to those described by Jelinek et al., Science 259:1614 (1993).

Those of skill in the art can devise various methods to measure the ability of Zlipo1/glycodelin polypeptides, with or without a Zlipo1/glycodelin ligand, to induce physiological effects. For example, human postmortem vomeronasal membranes for signal transduction studies can be isolated employing a method described for rodent vomeronasal membrane preparations (Kroner et al., Neuroreport 7:2989 (1996)). Moreover, stimulation experiments and second messenger assays, performed with recombinant Zlipo1/glycodelin alone or in combination with ligand, can be carried out employing the method described by Krieger et al., J. Biol. Chem. 274:4655 (1999). Formulations of Zlipo1/glycodelin alone or in combination with ligand, can also be assayed on vomeronasal organs of human volunteers as described by Monti-Bloch and Grosser, J. Steroid Biochem. 39:573 (1991), and by Grosser et al., Psychoneuroendocrinology 25:289 (2000). These assays can be used to assess changes in the electrophysiological output of the vomeronasal organ, as well as alternations in autonomic functions, and changes in transient feelings and moods. Alternations of hypothalamic functions, such as satiety, energy balance, sexual motivation, anxiety and the like, can also be evaluated in test subjects using a variety of recognized standard test protocols. Useful formulations of Zlipo1/glycodelin can be conveniently delivered to vomeronasal organ by intranasal administration.

In addition, a behavioral assay for human nipple search pheromone activity can be carried out based on the method described for the rabbit (Keil et al., Physiol. Behav. 47:525 (1990)). Test samples on a glass rod are presented approximately five millimeters from the nasal cavity of the test subject. A positive response is recorded if the presentation elicits a clear, search-like head movement or gasping of the rod within ten seconds. Alternatively, a “two-choice odor-preference test” similar to the one described by Makin and Porter, Child Develop 60:803 (1989), can be used to detect a nipple search pheromone activity. In that assay, infants oriented preferentially to an odorized breast pad from a nursing woman. Other behavioral assays that can be used detect a nipple search pheromone activities are described by Winberg and Porter, Acta Paediatr. 87:6 (1998), Porter and Winberg, Neurosci. Behav. Rev. 23:439 (1999), and Blass and Teicher, Science 210:15 (1980).

In another approach, a Zlipo1/glycodelin polypeptide or fusion protein can be immobilized onto the surface of a receptor chip of a biosensor instrument (BIACORE, Biacore AB; Uppsala, Sweden) to detect the presence of a Zlipo1/glycodelin target, such as a cognate receptor or ligand. The use of this instrument is disclosed, for example, by Karlsson, Immunol. Methods 145:229 (1991). In brief, a Zlipo1/glycodelin polypeptide or fusion protein is covalently attached, using amine or sulfhydryl chemistry, to dextran fibers that are attached to gold film within a flow cell. A test sample is then passed through the cell. If a Zlipo1/glycodelin target molecule is present in the sample, it will bind to the immobilized polypeptide or fusion protein, causing a change in the refractive index of the medium, which is detected as a change in surface plasmon resonance of the gold film. This system allows the determination on- and off-rates, from which binding affinity can be calculated, and assessment of the stoichiometry of binding, as well as the kinetic effects of Zlipo1/glycodelin mutation.

7. Therapeutic Uses of the Lipocalin Polypeptides

The present invention includes the use of proteins, polypeptides, and peptides having Zlipo1/glycodelin activity (such as Zlipo1/glycodelin polypeptides, anti-idiotype anti-Zlipo1/glycodelin antibodies, and Zlipo1/glycodelin fusion proteins) to a subject who lacks an adequate amount of this polypeptide. The Zlipo1/glycodelin molecules described herein can be administered, with or without a cognate phermone ligand, to any subject in need of treatment, and the present invention contemplates both veterinary and human therapeutic uses. Illustrative subjects include mammalian subjects, such as farm animals, domestic animals, and human patients.

For example, the nasal administration of phermones to human subjects affects the hypothalamus, which in turn, affects the function of the autonomic nervous system and a variety of behavioral and physiological phenomena, including anxiety, premenstrual stress, aggression, hunger, blood pressure, and other functions mediated by the hypothalamus (see, for example, Berliner et al., U.S. Pat. No. 5,969,168).

Sobel, international patent publication No. WO00/23141, describes a device for electrical stimulation of the human vomeronasal organ to affect hypothalamic activity, to regulate hormone levels, to treat diseases such as prostate cancer, to treat reproductive disorders, and to treat affective disorders. The administration of Zlipo1 or glycodelin provides an alternative means for stimulating the vomeronasal organ.

Generally, the dosage of administered polypeptide, protein or peptide will vary depending upon such factors as the subject's age, weight, height, sex, general medical condition and previous medical history. Typically, it is desirable to provide the recipient with a dosage of a molecule having Zlipo1/glycodelin activity, which is in the range of from about 1 pg/kg to 10 mg/kg (amount of agent/body weight of subject), although a lower or higher dosage also may be administered as circumstances dictate.

Molecules having Zlipo1/glycodelin activity can be administered to a subject by oral, dermal, mucosal-membrane, pulmonary, and transcutaneous routes. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)).

Conveniently, molecules having Zlipo1/glycodelin activity can be administered by an intranasal route. A liopcalin-containing spray for administration to the nasal mucosa of a subject may comprise a solution of Zlipo1 or glycodelin, or a pharmaceutically acceptable salt thereof, in a pharmaceutically acceptable solvent (e.g., phosphate-buffered saline). Such a spray may further comprise a viscosity agent, such as cellulose, a substituted cellulose, or a pharmaceutically acceptable oil emulsion. The present invention also includes liposomal compositions suitable for the aerosol or spray delivery of Zlipo1 or glycodelin to a subject. Such a composition may comprise Zlipo1/glycodelin, and optionally an additional supplement, in phospholipid liposomes, and a carrier. Ilustrative liposomes have a diameter between about 20 nm and 10 microns. Additional supplements include anti-microbial agents and antioxidants. These liposomal compositions can be administered in a variety of aerosol or pump spray administration devices, such as pump actuated sprayers, atomizers and nebulizers that are known to those in the art

The feasibility of an intranasal delivery of a polypeptide is exemplified by one mode of insulin administration (see, for example, Hinchcliffe and illum, Adv. Drug Deliv. Rev. 35:199 (1999)). Dry or liquid particles comprising Zlipo1/glycodelin can be prepared and inhaled with the aid of dry-powder dispersers, liquid aerosol generators, or nebulizers (e.g., Pettit and Gombotz, TIBTECH 16:343 (1998); Patton et al., Adv. Drug Deliv. Rev. 35:235 (1999)). This approach is illustrated by the AERX diabetes management system, which is a hand-held electronic inhaler that delivers aerosolized insulin into the lungs.

As an alternative, Zlipo1/glycodelin can be administered to a subject using a neuroepithelial sample delivery system, which is exemplified by the device described by Monti-Bloch, U.S. Pat. No. 5,303,703.

Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850 (1995)). Transdermal delivery using electroporation provides another means to administer Zlipo1/glycodelin (Potts et al., Pharm. Biotechnol. 10:213 (1997)).

A molecule having Zlipo1/glycodelin activity can also be administered to a subject by intravenous, intraarterial, intraperitoneal, intramuscular, subcutaneous, intrapleural, or intrathecal routes, or by perfusion through a regional catheter. When administering therapeutic proteins by injection, the administration may be by continuous infusion or by single or multiple boluses.

A pharmaceutical composition comprising a protein, polypeptide, or peptide having Zlipo1/glycodelin activity can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient subject. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, molecules having Zlipo1/glycodelin activity and a pharmaceutically acceptable carrier are administered to a subject in a therapeutically effective amount. A combination of a protein, polypeptide, or peptide having Zlipo1/glycodelin activity and a pharmaceutically acceptable carrier is said to be administered in a “therapeutically effective amount” if the amount administered is physiologically significant. An agent is physiologically significant if its presence results in a detectable change in the physiology or behavior of a recipient subject. One example of a modification of behavior is a reduction of anxiety.

A pharmaceutical composition comprising molecules having Zlipo1/glycodelin activity can be furnished in liquid form, or in solid form. Liquid forms, including liposome-encapsulated formulations, are illustrated by injectable solutions and oral suspensions. Exemplary solid forms include capsules, tablets, and controlled-release forms, such as a miniosmotic pump or an implant. Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

The present invention also contemplates the use of chemically modified Zlipo1/glycodelin compositions, in which the polypeptide is linked with a polymer. Typically, the polymer is water soluble so that the Zlipo1/glycodelin conjugate does not precipitate in an aqueous environment, such as a physiological environment. An example of a suitable polymer is one that has been modified to have a single reactive group, such as an active ester for acylation, or an aldehyde for alkylation, In this way, the degree of polymerization can be controlled. An example of a reactive aldehyde is polyethylene glycol propionaldehyde, or mono-(C₁-C₁₀) alkoxy, or aryloxy derivatives thereof (see, for example, Harris, et al., U.S. Pat. No. 5,252,714). The polymer may be branched or unbranched. Moreover, a mixture of polymers can be used to produce Zlipo1/glycodelin conjugates.

Zlipo1/glycodelin conjugates used for therapy should preferably comprise pharmaceutically acceptable water-soluble polymer moieties. Suitable water-soluble polymers include polyethylene glycol (PEG), monomethoxy-PEG, mono-(C₁-C₁₀)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A Zlipo1/glycodelin conjugate can also comprise a mixture of such water-soluble polymers. Anti-Zlipo1/glycodelin antibodies or anti-idiotype antibodies can also be conjugated with a water-soluble polymer.

Zlipo1/glycodelin pharmaceutical compositions can be supplied as a kit comprising a container that comprises Zlipo1/glycodelin. Zlipo1/glycodelin can be provided in the form of an injectable solution for single or multiple doses, as a sterile powder that will be reconstituted before injection, or in a device suitable for intranasal administration. Such a kit may further comprise written information on indications and usage of the pharmaceutical composition. Moreover, such information may include a statement that the Zlipo1/glycodelin composition is contraindicated in subjects with known hypersensitivity to Zlipo1/glycodelin.

In addition, compositions comprising at least one of Zlipo1 and glycodelin can be used as additives for baby formulae. For this purpose, Zlipo1 or glycodelin can be conveniently provided as lyophilized polypeptides, or in the form of a concentrated solution.

8. Therapeutic Uses of Zlipo1 and Glycodelin Nucleotide Sequences

The present invention includes the use of nucleotide sequences to provide Zlipo1/glycodelin to a subject in need of such treatment. In addition, a therapeutic expression vector can be provided that inhibits Zlipo1/glycodelin gene expression, such as an anti-sense molecule, a ribozyme, or an external guide sequence molecule.

There are numerous approaches to introduce a Zlipo1/glycodelin gene to a subject, including the use of recombinant host cells that express Zlipo1/glycodelin, delivery of naked nucleic acid encoding Zlipo1/glycodelin, use of a cationic lipid carrier with a nucleic acid molecule that encodes Zlipo1/glycodelin, and the use of viruses that express Zlipo1/glycodelin, such as recombinant retroviruses, recombinant adeno-associated viruses, recombinant adenoviruses, and recombinant Herpes simplex viruses (see, for example, Mulligan, Science 260:926 (1993), Rosenberg et al., Science 242:1575 (1988), LaSalle et al., Science 259:988 (1993), Wolff et al., Science 247:1465 (1990), Breakfield and Deluca, The New Biologist 3:203 (1991)). In an ex vivo approach, for example, cells are isolated from a subject, transfected with a vector that expresses a Zlipo1/glycodelin gene, and then transplanted into the subject.

In order to effect expression of a Zlipo1/glycodelin gene, an expression vector is constructed in which a nucleotide sequence encoding a Zlipo1/glycodelin gene is operably linked to a core promoter, and optionally a regulatory element, to control gene transcription. The general requirements of an expression vector are described above.

Alternatively, a Zlipo1/glycodelin gene can be delivered using recombinant viral vectors, including for example, adenoviral vectors (e.g., Kass-Eisler et al., Proc. Nat'l Acad. Sci. USA 90:11498 (1993), Kolls et al., Proc. Nat'l Acad. Sci. USA 91:215 (1994), Li et al., Hum. Gene Ther. 4:403 (1993), Vincent et al., Nat. Genet. 5:130 (1993), and Zabner et al., Cell 75:207 (1993)), adenovirus-associated viral vectors (Flotte et al., Proc. Nat'l Acad. Sci. USA 90:10613 (1993)), alphaviruses such as Semliki Forest Virus and Sindbis Virus (Hertz and Huang, J. Vir. 66:857 (1992), Raju and Huang, J. Vir. 65:2501 (1991), and Xiong et al., Science 243:1188 (1989)), herpes viral vectors (e.g., U.S. Pat. Nos. 4,769,331, 4,859,587, 5,288,641 and 5,328,688), parvovirus vectors (Koering et al., Hum. Gene Therap. 5:457 (1994)), pox virus vectors (Ozaki et al., Biochem. Biophys. Res. Comm. 193:653 (1993), Panicali and Paoletti, Proc. Nat'l Acad. Sci. USA 79:4927 (1982)), pox viruses, such as canary pox virus or vaccinia virus (Fisher-Hoch et al., Proc. Nat'l Acad. Sci. USA 86:317 (1989), and Flexner et al., Ann. N.Y. Acad. Sci. 569:86 (1989)), and retroviruses (e.g., Baba et al., J. Neurosurg 79:729 (1993), Ram et al., Cancer Res. 53:83 (1993), Takamiya et al., J. Neurosci. Res 33:493 (1992), Vile and Hart, Cancer Res. 53:962 (1993), Vile and Hart, Cancer Res. 53:3860 (1993), and Anderson et al., U.S. Pat. No. 5,399,346). Within various embodiments, either the viral vector itself, or a viral particle which contains the viral vector may be utilized in the methods and compositions described below.

As an illustration of one system, adenovirus, a double-stranded DNA virus, is a well-characterized gene transfer vector for delivery of a heterologous nucleic acid molecule (for a review, see Becker et al., Meth. Cell Biol. 43:161 (1994); Douglas and Curiel, Science & Medicine 4:44 (1997)). The adenovirus system offers several advantages including: (i) the ability to accommodate relatively large DNA inserts, (ii) the ability to be grown to high-titer, (iii) the ability to infect a broad range of mammalian cell types, and (iv) the ability to be used with many different promoters including ubiquitous, tissue specific, and regulatable promoters. In addition, adenoviruses can be administered by intravenous injection, because the viruses are stable in the bloodstream.

Using adenovirus vectors where portions of the adenovirus genome are deleted, inserts are incorporated into the viral DNA by direct ligation or by homologous recombination with a co-transfected plasmid. In an exemplary system, the essential E1 gene is deleted from the viral vector, and the virus will not replicate unless the E1 gene is provided by the host cell. When intravenously administered to intact animals, adenovirus primarily targets the liver. Although an adenoviral delivery system with an E1 gene deletion cannot replicate in the host cells, the host's tissue will express and process an encoded heterologous protein. Host cells will also secrete the heterologous protein if the corresponding gene includes a secretory signal sequence. Secreted proteins will enter the circulation from tissue that expresses the heterologous gene (e.g., the highly vascularized liver).

Moreover, adenoviral vectors containing various deletions of viral genes can be used to reduce or eliminate immune responses to the vector. Such adenoviruses are E1-deleted, and in addition, contain deletions of E2A or E4 (Lusky et al., J. Virol. 72:2022 (1998); Raper et al., Human Gene Therapy 9:671 (1998)). The deletion of E2b has also been reported to reduce immune responses (Amalfitano et al., J. Virol. 72:926 (1998)). By deleting the entire adenovirus genome, very large inserts of heterologous DNA can be accommodated. Generation of so called “gutless” adenoviruses, where all viral genes are deleted, are particularly advantageous for insertion of large inserts of heterologous DNA (for a review, see Yeh. and Pernicaudet, FASEB J. 11:615 (1997)).

High titer stocks of recombinant viruses capable of expressing a therapeutic gene can be obtained from infected mammalian cells using standard methods. For example, recombinant HSV can be prepared in Vero cells, as described by Brandt et al., J. Gen. Virol. 72:2043 (1991), Herold et al., J. Gen. Virol. 75:1211 (1994), Visalli and Brandt, Virology 185:419 (1991), Grau et al., Invest. Ophthalmol. Vis. Sci. 30:2474 (1989), Brandt et al., J. Virol. Meth. 36:209 (1992), and by Brown and MacLean (eds.), HSV Virus Protocols (Humana Press 1997).

Alternatively, an expression vector comprising a Zlipo1/glycodelin gene can be introduced into a subject's cells by lipofection in vivo using liposomes. Synthetic cationic lipids can be used to prepare liposomes for in vivo transfection of a gene encoding a marker (Felgner et al., Proc. Nat'l Acad. Sci. USA 84:7413 (1987); Mackey et al., Proc. Nat'l Acad. Sci. USA 85:8027 (1988)). The use of lipofection to introduce exogenous genes into specific organs in vivo has certain practical advantages. Liposomes can be used to direct transfection to particular cell types, which is particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

Electroporation is another alternative mode of administration of a Zlipo1/glycodelin nucleic acid molecules. For example, Aihara and Miyazaki, Nature Biotechnology 16:867 (1998), have demonstrated the use of in vivo electroporation for gene transfer into muscle.

In an alternative approach to gene therapy, a therapeutic gene may encode a Zlipo1/glycodelin anti-sense RNA that inhibits the expression of Zlipo1/glycodelin. Methods of preparing anti-sense constructs are known to those in the art. See, for example, Erickson et al., Dev. Genet. 14:274 (1993) [transgenic mice], Augustine et al., Dev. Genet. 14:500 (1993) [murine whole embryo culture], and Olson and Gibo, Exp. Cell Res. 241:134 (1998) [cultured cells]. Suitable sequences for Zlipo1/glycodelin anti-sense molecules can be derived from the nucleotide sequences of Zlipo1/glycodelin disclosed herein.

Alternatively, an expression vector can be constructed in which a regulatory element is operably linked to a nucleotide sequence that encodes a ribozyme. Ribozymes can be designed to express endonuclease activity that is directed to a certain target sequence in a mRNA molecule (see, for example, Draper and Macejak, U.S. Pat. No. 5,496,698, McSwiggen, U.S. Pat. No. 5,525,468, Chowrira and McSwiggen, U.S. Pat. No. 5,631,359, and Robertson and Goldberg, U.S. Pat. No. 5,225,337). In the context of the present invention, ribozymes include nucleotide sequences that bind with Zlipo1/glycodelin mRNA.

In another approach, expression vectors can be constructed in which a regulatory element directs the production of RNA transcripts capable of promoting RNase P-mediated cleavage of mRNA molecules that encode a Zlipo1/glycodelin gene. According to this approach, an external guide sequence can be constructed for directing the endogenous ribozyme, RNase P, to a particular species of intracellular mRNA, which is subsequently cleaved by the cellular ribozyme (see, for example, Altman et al., U.S. Pat. No. 5,168,053, Yuan et al., Science 263:1269 (1994), Pace et al., international publication No. WO 96/18733, George et al., international publication No. WO 96/21731, and Werner et al., international publication No. WO 97/33991). Preferably, the external guide sequence comprises a ten to fifteen nucleotide sequence complementary to Zlipo1/glycodelin mRNA, and a 3′-NCCA nucleotide sequence, wherein N is preferably a purine. The external guide sequence transcripts bind to the targeted mRNA species by the formation of base pairs between the mRNA and the complementary external guide sequences, thus promoting cleavage of mRNA by RNase P at the nucleotide located at the 5′-side of the base-paired region.

In general, the dosage of a composition comprising a therapeutic vector having a Zlipo1/glycodelin nucleotide acid sequence, such as a recombinant virus, will vary depending upon such factors as the subject's age, weight, height, sex, general medical condition and previous medical history. Suitable routes of administration of therapeutic vectors include intravenous injection, intraarterial injection, intraperitoneal injection, and intramuscular injection.

A composition comprising viral vectors, non-viral vectors, or a combination of viral and non-viral vectors of the present invention can be formulated according to known methods to prepare pharmaceutically useful compositions, whereby vectors or viruses are combined in a mixture with a pharmaceutically acceptable carrier. As noted above, a composition, such as phosphate-buffered saline is said to be a “pharmaceutically acceptable carrier” if its administration can be tolerated by a recipient subject. Other suitable carriers are well-known to those in the art (see, for example, Remington's Pharmaceutical Sciences, 19th Ed. (Mack Publishing Co. 1995), and Gilman's the Pharmacological Basis of Therapeutics, 7th Ed. (MacMillan Publishing Co. 1985)).

For purposes of therapy, a therapeutic gene expression vector, or a recombinant virus comprising such a vector, and a pharmaceutically acceptable carrier are administered to a subject in a therapeutically effective amount. A combination of an expression vector (or virus) and a pharmaceutically acceptable carrier is said to be administered in a “therapeutically effective amount” if the amount administered is physiologically significant An agent is physiologically significant if its presence results in a detectable change in the physiology or behavior of a recipient subject. One example of a modification of behavior is a reduction of anxiety.

When the subject treated with a therapeutic gene expression vector or a recombinant virus is a human, then the therapy is preferably somatic cell gene therapy. That is, the preferred treatment of a human with a therapeutic gene expression vector or a recombinant virus does not entail introducing into cells a nucleic acid molecule that can form part of a human germ line and be passed onto successive generations (i.e., human germ line gene therapy).

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of identifying the presence of a Zlipo1 receptor or a glycodelin receptor in a test sample, comprising: (a) contacting the test sample with a Zlipo1 or glycodelin polypeptide that comprises the amino acid sequence of SEQ ID NO:2 or the amino acid sequence of SEQ ID NO:4, and (b) detecting the binding of the polypeptide to the cognate receptor in the test sample.

2. The method of claim 1, wherein the test sample comprises cultured cells.

3. The method of claim 2, wherein the cultured cells are recombinant host cells transfected with a cDNA library prepared from vomeronasal tissue.

4. The method of claim 3, wherein the cDNA library is prepared from human vomeronasal tissue.

5. The method of claim 2, wherein the cultured cells are recombinant host cells transfected with a cDNA library prepared from main olfactory epithelium tissue.

6. The method of claim 5, wherein the cDNA library is prepared from human main olfactory epithelium tissue.

7. The method of claim 1, wherein the Zlipo1 or glycodelin is contacted with a cell membrane preparation.

8. The method of claim 7, wherein the cell membrane preparation is obtained from recombinant host cells transfected with a cDNA library prepared from vomeronasal tissue.

9. The method of claim 8, wherein the cDNA library is prepared from human vomeronasal tissue.

10. The method of claim 7, wherein the cell membrane preparation is obtained from recombinant host cells transfected with a cDNA library prepared from main olfactory epithelium tissue.

11. The method of claim 10, wherein the cultured cells are recombinant host cells transfected with a cDNA library prepared from main olfactory epithelium tissue.

12. A method of identifying the presence of a Zlipo1 ligand or a glycodelin ligand in a test sample, comprising: (a) contacting the test sample with a Zlipo1 or glycodelin polypeptide that comprises the amino acid sequence of SEQ ID NO:2 or the amino acid sequence of SEQ ID NO:4, and (b) detecting the binding of the polypeptide to ligand in the test sample.
